(19) United States
(12) Patent Application Publication
(10) Pub. No.: US 2009/0148937 A1
(43) Pub. Date: Jun. 11, 2009

(54) MICRO-FLUIDIC SYSTEM COMPRISING AN EXPANDED CHANNEL

(75) Inventors: Thomas Schnelle, Berlin (DE); Torsten Müller, Berlin (DE); Annette Pfennig, Berlin (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: PERKINELMER CELLULAR TECHNOLOGIES,, Hamburg (DE)

(21) Appl. No.: 11/719,618

(22) PCT Filed: Nov. 17, 2005
(86) PCT No.: PCT/EP05/56045

§ 371 Date: Jun. 19, 2007

(30) Foreign Application Priority Data
Nov. 18, 2004 (DE) 10 2004 055 662.8

(51) Int. Cl. C12N 5/06; G01N 15/00; G01N 27/26; B03B 5/00

(52) U.S. Cl. 435/325; 73/61.71; 204/643; 209/132

(57) ABSTRACT

The invention relates to a micro-fluidic system including a fluid medium channel that holds a fluid medium containing particles in suspension. According to the invention, the fluid medium channel has an expanded section with an enlarged cross-section along part of its length, in order to reduce the flow speed to allow the analysis of the particles.

The invention relates to a microfluidic system, in particular for a cell sorter, comprising a carrier flow channel for accommodating a carrier flow containing particles suspended therein, according to the preamble of claim 1.

Such a microfluidic system is known for example from DE 103 20 956 A1 and can be used in a cell sorter in order to analyze biological cells and sort the cells into one of a plurality of output channels depending on the result of the analysis. To this end, the known microfluidic system comprises a carrier flow channel for accommodating a carrier flow containing particles suspended therein, wherein the carrier flow channel branches into a plurality of output channels, into which the biological cells are sorted. Furthermore, a measurement station is arranged in the carrier flow channel, which measurement station analyses the suspended biological cells, for example by carrying out a light transmission measurement, a fluorescence measurement or impedance spectroscopy. However, it is also possible that the measurement station measures the deformation of the suspended particles or the speed of rotation thereof or else an electrical or magnetic parameter. The analysis of the suspended biological cells in the measurement station requires that the biological cells to be analyzed are spatially fixed or at least significantly slowed down during the analysis. The known microfluidic system therefore has a field cage for fixing the biological cells to be analyzed in the carrier flow channel, which field cage consists of an electrophoretically acting electrode arrangement and stops the biological cells suspended in the carrier flow by means of suitable electrical actuation so that the measurement station can analyze the cells in the resting state.

The disadvantages of the known microfluidic system described above include the quantitatively unsatisfactory throughput and the high stress on the biological cells.

The object of the invention is therefore to increase the throughput of biological cells in such a microfluidic system.

This object is achieved by an inventive microfluidic system according to claim 1.

The invention is based on the newly obtained finding that the throughput of biological cells in the known microfluidic system described above is limited on the one hand by the maximum permissible detection speed of the measurement station and on the other hand by the maximum permissible electrical actuation of the field cage.

This is because, in order to carry out an analysis in the measurement station, the suspended biological cells must not exceed a certain flow speed. The field cage therefore slows down the biological cells from the normal flow speed in the carrier flow channel until the flow speed of the cells to be analyzed falls below the maximum permissible detection speed of the measurement station.

However, this slowing-down of the cells to be analyzed is possible only to a limited extent since the electrical voltage applied to the field cage in order to slow down or stop the cells cannot be increased at will since the cells may otherwise be thermally or electrically damaged.

Increasing the flow speed in the carrier flow channel can therefore be used to increase the quantitative throughput only so far until, despite the maximum permissible electrical actuation of the field cage, the maximum permissible detection speed of the measurement station is reached.

The invention therefore encompasses the general technical teaching that the carrier flow channel has over part of its length a channel widening with a widened channel cross section. Depending on the ratio of the channel cross sections before of the channel widening and in the channel widening, the channel widening leads to a corresponding reduction in the flow speed, as a result of which the slowing-down by the field cage of the cells to be analyzed can be assisted or even replaced.

The channel widening of the carrier flow channel according to the invention offers the advantage that the flow speed in the carrier flow channel outside the channel widening and thus also the quantitative throughput of biological cells or other particles can be increased, without the particles to be analyzed at the measurement station exceeding the maximum detection speed.

A further advantage of the channel widening consists in that the field cage or the measurement station can be arranged further away from the channel edge. This is particularly advantageous in the case of high-resolution fluorescence measurements, which may be hindered by fluorescent channel materials or adhesives.

In the preferred exemplary embodiment of the invention, at least one measurement station for analyzing the cells or other particles suspended in the carrier flow is arranged in the region of the channel widening. The measurement station per se may be designed in a conventional manner, as described for example in the document laid open to inspection DE 103 20 956 A1 already cited above, and therefore the content of said publication with regard to the design and function of the measurement station is hereby fully incorporated into the present description.

The scope of the invention also includes the possibility that at least one manipulation device for manipulating the suspended particles is arranged in the region of the channel widening, wherein the reduced flow speed in the region of the channel widening facilitates the manipulation of the suspended particles. By way of example, a sorting device (e.g. a dielectric switch) may be arranged as the manipulation device in the region of the channel widening, which sorting device sorts different particles (e.g. red and white blood cells). The manipulation device may also be a retaining device which stops the suspended particles when suitably actuated. However, it is also possible as an alternative that the manipulation device carries out a manipulation in the narrower sense, by stretching the particles or by forming pairs (e.g. by means of electrofusion), as known per se. The manipulation device may in this case be for example a laser or a laser tweezer or a dielectrophoretic electrode arrangement.

The channel widening is in this case limited in the flow direction preferably to the region of the measurement station or of the manipulation device, since only there is a reduction in flow speed necessary in order to allow analysis in the measurement station or manipulation of the particles.

Furthermore, in one preferred exemplary of embodiment of the invention, the measurement station has a predefined maximum permissible detection speed, up to which the measurement station can analyze the particles suspended in the carrier flow. The carrier flow on the other hand has a flow speed which is below the maximum detection speed in the region of the channel widening and above the maximum detection speed outside the channel widening. The channel widening thus leads in this case to a reduction in the flow speed until it is below the maximum permissible detection speed of the measurement station, so that the flow speed in the carrier flow channel before the channel widening can accordingly be increased, as a result of which the quantitative throughput of particles is increased.

Moreover, the reduction in the flow speed in the region of the channel widening offers the possibility that there is no need for a field cage or any other fixing device for stopping the particles during the analysis.

One significant advantage of the use of microchannels for analyzing biological samples (e.g. cells), in particular for analyzing their reaction to the addition of agents (e.g. pharmaceutically active or cell-differentiating substances), consists in that only very small volumes are required. This is of great importance in the case of active agent screening. However, the small channel dimensions place strict limits on parallel analysis. If one channel widening contains a plurality of manipulation elements in which individual cells or cell aggregates can be held, the advantages of the small substance quantities can be combined with the ability to carry out parallel analysis.

However, the invention is not restricted to those embodiments in which no field cage is arranged in the region of the channel widening. Rather, it is also possible that the slowing-down or fixing of the particles to be analyzed during the analysis takes place jointly by the channel widening and a field cage, as a result of which the flow speed in the carrier flow channel before the channel widening and thus the throughput of particles can be increased still further.

The field cage is preferably arranged in the region of the measurement station, in order to slow down or even fix the particles for analysis by the measurement station.

However, it is also possible that the field cage with its electrodes at the same time forms the measurement station, so that the field cage and the measurement station are integrated in one bifunctional component. Such bifunctional electrode arrangements are described for example in the patent application DE 10 2004 017 482.2, and therefore the content of said patent application is hereby fully incorporated into the present description.

The above-described bifunctional integration is not restricted to the combination of a dielectric field cage with a measurement station. For example, it is also possible to integrate the measurement station with a manipulation device (e.g. a laser or a laser tweezer) in one component, wherein the manipulation device may also operate magnetically.

The channel cross section of the carrier flow channel is preferably widened by 5% to 400% in the region of the channel widening compared to the region outside the channel widening, wherein within the scope of the invention any intermediate values are possible and a range from 10% to 500%, preferably 10% to 300%, is particularly advantageous.

In the preferred examples of embodiments of the invention, the carrier flow channel branches into a plurality of output channels in a branching region located downstream behind the channel widening, into which output channels the particles to be analyzed can be sorted. Such a branching is known for example from the document DE 103 20 956 A1 already cited above, and therefore the content thereof with regard to the design of the microfluidic system in the branching region is hereby fully incorporated into the present description.

A sorting device is preferably arranged in the branching region, which sorting device sorts the suspended particles into one of the output channels depending on the actuation of the sorting device, wherein such a sorting device is also known from the document DE 103 20 956 A1 already cited above.

Furthermore, a centering device is preferably arranged in at least one of the output channels, which centering device centers the suspended particles in the output channel and thereby prevents particles from settling in the output channels due to the force of gravity.

Moreover, at least one sheath flow channel preferably opens into at least one of the output channels, as is also known per se.

A retaining device (“hook”) may be located in the carrier flow channel upstream before the measurement station, which retaining device stops the suspended particles or allows them to pass, depending on its actuation. This offers the possibility of stopping the particles to be analyzed upstream before the measurement station and supplying them to the measurement station in a targeted manner.

The sorting device (“switch”), the centering device (“funnel”), the field cage (“cage”) and/or the retaining device (“hook”) in this case preferably comprise a dielectrophoretically acting electrode arrangement. Such dielectrophoretically acting electrode arrangements are known for example from Müller, T. et al.: “A 3-D microelectrode system for handling and caging single cells and particles”, Biosensors and Bioelectronics 14 (1999), 247-256, and therefore the content of said publication is hereby fully incorporated into the present description.

However, instead of dielectrophoretically acting electrode arrangements, it is also possible within the scope of the invention to use other electrokinetic forces which are based for example on electrophoresis or electroosmosis. To this end, an additional channel may be provided which is connected to the carrier flow channel and runs essentially transversely to the carrier flow channel, wherein the additional channel is supplied with DC voltage signals in order to deflect the particles. Furthermore, it is also possible for the particles to be manipulated magnetically or by means of lasers (e.g. by means of a laser tweezer). The individual particles may also be stretched, as described for example in DE 103 52 416, and therefore the content of said document is hereby fully incorporated into the present description.

It is also possible that the sorting device and/or the measurement station is arranged eccentrically in the carrier flow channel. The eccentric arrangement of the sorting device in front of the mouth opening of one of the output channels offers the possibility that the particles to be sorted automatically flow into the respective output channel without active actuation of the sorting device, so that the sorting device has to be actively actuated only for sorting into one of the other output channels.

In the microfluidic system according to the invention, the channel widening may be one-dimensional so that for example only the width of the carrier flow channel is enlarged in the region of the channel widening, while the height of the carrier flow channel remains constant.

However, it is also possible that the channel widening is two-dimensional, so that both the height and the width of the carrier flow channel are enlarged in the region of the channel widening.

Furthermore, it is advantageous if the microfluidic system according to the invention is integrated on a chip.

Moreover, the channel widening can also be achieved in that the carrier flow channel branches into a plurality of parallel subchannels upstream before the channel widening, which subchannels recombine again downstream behind the channel widening. The total cross section of the individual subchannels is in this case preferably greater than the cross section of the carrier flow channel outside the channel widening, so that in this case too the flow speed is reduced in the region of the channel widening.

It is also possible that a plurality of channel widenings is arranged one behind the other in the carrier flow channel. This may be particularly useful when a plurality of measurement stations are arranged one behind the other in the carrier flow channel. The individual channel widenings are then preferably arranged in each case at the location of the measurement stations, in order to reduce the flow speed of the suspended particles at that point and thereby allow a measurement.

It is also possible that the microfluidic system according to the invention comprises a plurality of parallel or branching carrier flow channels, in which at least one channel widening is arranged in each case.

Furthermore, the lowering of the flow speed according to the invention in the region of the channel widening is useful not only for analyzing the particles but also for the manipulation thereof, such as for example for the targeted formation of pairs. It is therefore also possible within the scope of the invention that a manipulation device is arranged in the region of the channel widening.

It should also be mentioned that the microfluidic system according to the invention can advantageously be used in a cell sorter.

By means of a suitable channel widening, it is also possible without changing the pump rate to extend/adjust the residence time of the microobjects, as is necessary within the context of a required assay incubation time. As a result, a coupling is achieved between a continuous pump rate and a discontinuous time regime.

The invention also encompasses the novel use of such a microfluidic system in medical or pharmaceutical research, in diagnostics or in forensic medicine.

Moreover, the invention also encompasses the use of a microfluidic system according to the invention for separating different cell types from one another, such as in particular apoptotic and necrotic cells, cells with different expression patterns and/or stem cells. Cells or particles in general which have a different size and/or a different morphology can also be sorted in the microfluidic system according to the invention.

It should also be mentioned that the term “particle” used within the context of the invention should be understood in a general manner and is not restricted to individual biological cells. Rather, this term also encompasses synthetic or biological particles, with particular advantages being obtained if the particles comprise biological materials, that is to say for example biological cells, cell groups, cell components or biologically relevant macromolecules, in each case optionally in association with other biological particles or synthetic carrier particles. Synthetic particles may comprise solid particles, liquid particles bounded by the suspension medium, or multiphase particles which form a separate phase from the suspension medium in the carrier flow.

Finally, it should be mentioned that the term “microfluidic system” used within the context of the invention means that the carrier flow channel contains a volume which is preferably in the milliliter, microliter or nanoliter range. In the microfluidic system according to the invention, the volume of the carrier flow channel may therefore lie for example in the range from 0.01 nl to 10 ml or in the narrower range from 1 nl to 1 ml, wherein any intermediate values are possible.

Other advantageous further developments of the invention are characterized in the dependent claims or will be explained in more detail below together with the description of the preferred examples of embodiments with reference to the figures, in which:

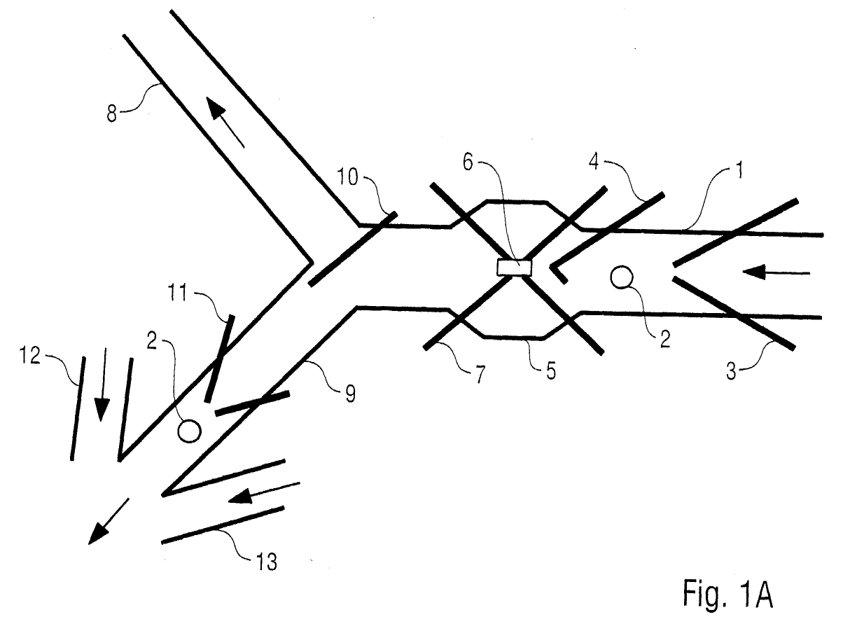

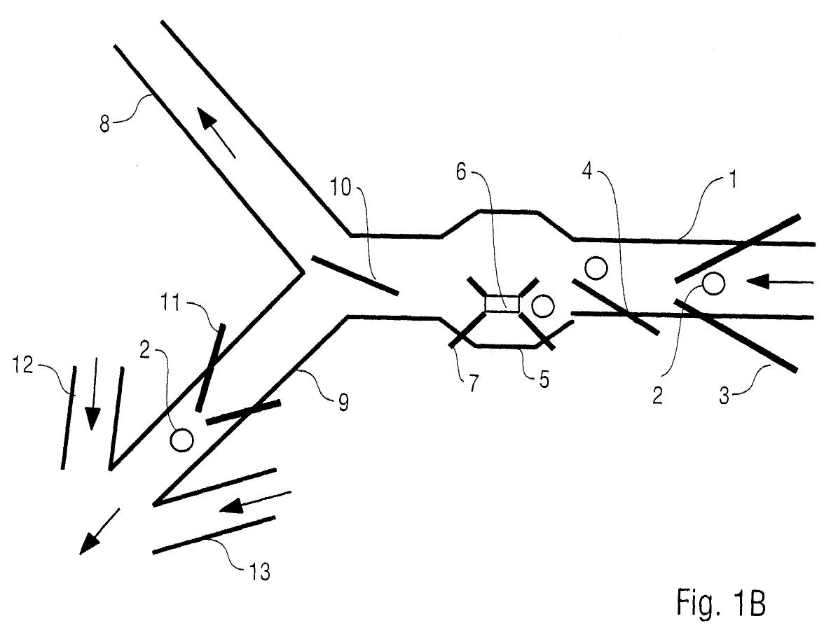

FIG. 1A shows a schematic diagram of a microfluidic system according to the invention with a channel widening and a field cage for jointly slowing down or fixing the particles to be analyzed,

FIG. 1B shows an alternative example of embodiment of a microfluidic system according to the invention with a channel widening and a non-central field cage for jointly slowing down or fixing the particles to be analyzed,

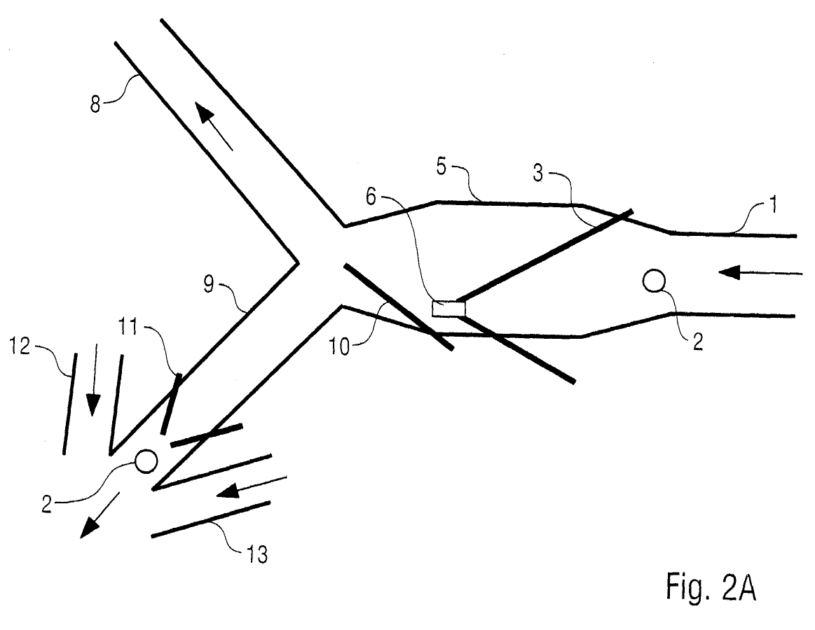

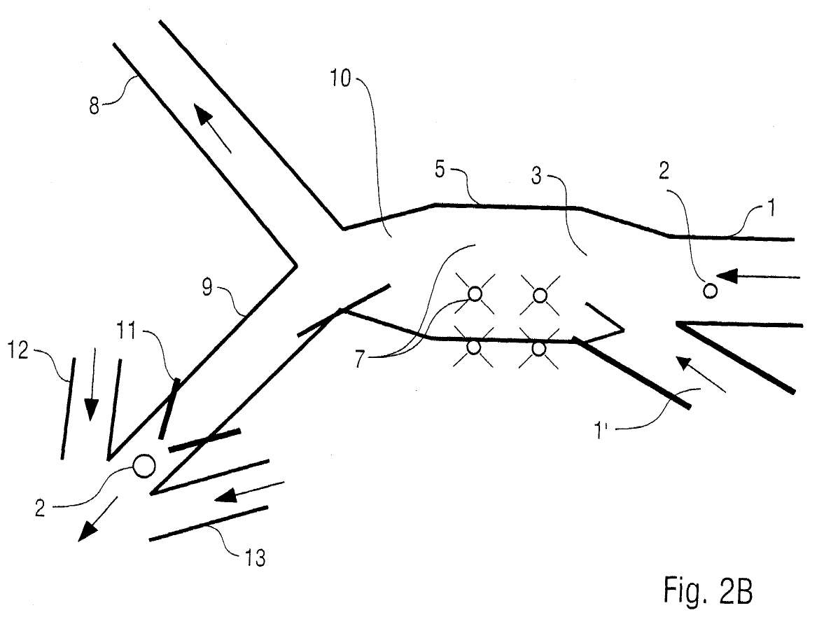

FIG. 2A shows an alternative example of embodiment of a microfluidic system according to the invention with a channel widening for slowing down the particles to be analyzed, without an additional field cage, and

FIG. 2B shows an alternative example of embodiment of a microfluidic system according to the invention with a channel widening for fixing and loading the particles to be analyzed.

The microfluidic system shown in FIG. 1A is partially designed in a conventional manner, and therefore reference is additionally made to the publication Müller, T. et al.: “A 3-D microelectrode system for handling and caging single cells and particles”, Biosensors and Bioelectronics 14 (1999), 247-256 and to DE 103 20 956 A1.

The microfluidic system comprises a carrier flow channel 1, in which there flows a carrier flow containing particles 2 suspended therein, as known per se.

Located in the carrier flow channel 1 is a funnel-shaped, dielectrophoretically acting electrode arrangement 3 which centers the particles 2 suspended in the carrier flow in the carrier flow channel 1 and is therefore also referred to as a “funnel”.

Located downstream behind the electrode arrangement 3 is a further dielectrophoretically acting electrode arrangement 4 which can largely stop the particles 2 centered and lined up by the funnel-shaped electrode arrangement 3 and is therefore referred to as a “hook”.

The carrier flow channel 1 has a channel widening 5 downstream behind the hook-like electrode arrangement 4, wherein the channel cross section in the region of the channel widening 5 is enlarged by 50% compared to the channel cross section outside the channel widening 5. The channel widening 5 brings about a reduction in the flow speed in the region of the channel widening, which is important for the subsequent analysis of the particles 2, as will be described in more detail below.

Located in the region of the channel widening 5 is a measurement station 6 which analyses the particles 2. The measurement station 6 may in this case be designed in a conventional manner, as described in the two publications mentioned above, so that there is no need for a detailed description of the measurement station 6 at this point.

However, it should be mentioned that the measurement station 6 can analyze the particles 2 only if the flow speed of the particles 2 does not exceed a predefined maximum permissible detection speed.

Also arranged in the region of the channel widening 5 is a further dielectrophoretically acting electrode arrangement 7 which is designed in a cage-like manner and which can dielectrophoretically fix the particles 2 when suitably electrically actuated and is therefore referred to as a “cage”.

The channel widening 5 and the cage-like electrode arrangement 7 in this case act together with the aim of slowing down or stopping the particle 2 in the carrier flow channel 1 so that the measurement station 6 can analyze the particles 2.

Downstream behind the channel widening 5, the carrier flow channel branches into two output channels 8, 9, wherein arranged in the branching region of the two output channels 8, 9 is a further dielectrophoretically acting electrode arrangement 10 which acts as a particle switch and is therefore also referred to as the “switch”. The switch-like electrode arrangement 10 sorts the particles 2 into one of the two output channels 8, 9 depending on its actuation and depending on the result of the analysis carried out by the measurement station 6, as known per se.

Located in the output channel 9 is a further funnel-like, dielectrophoretically acting electrode arrangement 11 which centers the particles 2 in the output channel 9 and thereby prevents the particles 2 from sinking in the output channel 9 due to the force of gravity.

Furthermore, two sheath flow channels 12, 13 open into the output channel 9, as is also known per se.

The microfluidic system shown in FIG. 1B largely corresponds to the example of embodiment described above and shown in FIG. 1A, and so in order to avoid repetition reference is largely made to the above description in respect of FIG. 1A, with the same references being used below for corresponding components.

The particular features of this example of embodiment consist in that the measurement station 6 and the cage-like electrode arrangement 7 are not located centrally in the region of the channel widening 5 and moreover the hook-like electrode arrangement 4 in FIG. 1A has been replaced by the electrode arrangement 4 with the function of a particle switch. Advantageously, when the electrode arrangement 7 (“cage”) is loaded, more particles 2 can be transferred tightly past the cage-like electrode arrangement 7 into the output channel 8 (“waste”) on account of the channel widening 5, which reduces the risk of blockage of the channel. Moreover, the particles 2 which have been positively evaluated by means of the measurement station 6 pass into the desired output channel 9 without additional switching.

The example of embodiment of a microfluidic system according to the invention which is shown in FIG. 2A largely corresponds to the example of embodiment described above and shown in FIG. 1A, and so in order to avoid repetition reference is largely made to the above description in respect of FIG. 1A, with the same references being used below for corresponding components.

One particular feature of this example of embodiment consists in that no additional cage-like electrode arrangement 7 is arranged in the region of the channel widening 5, so that the slowing-down of the particles 2 for analysis by the measurement station 6 is brought about solely by the channel widening 5.

A further particular feature of this example of embodiment consists in that the channel widening 5 extends over a much greater length of the carrier flow channel 1 compared to the example of embodiment shown in FIG. 1.

A final particular feature of this example of embodiment consists in that the funnel-like electrode arrangement 3, the measurement station 6 and the switch-like electrode arrangement 10 are in this case arranged in the carrier flow channel 1 in the region of the channel widening 5 eccentrically in front of the mouth opening of the output channel 9. In this case, therefore, the switch-like electrode arrangement 10 has to be actuated only if the particles 2 are to be sorted into the output channel 8, whereas the particles 2 to be sorted automatically flow into the output channel 9 without any active actuation of the switch-like electrode arrangement 10.

The example of embodiment of a microfluidic system according to the invention which is shown in FIG. 2B largely corresponds to the example of embodiment described above and shown in FIG. 2A, and so in order to avoid repetition reference is largely made to the above description in respect of FIG. 2A, with the same references being used below for corresponding components.

One particular feature of this example of embodiment consists in that a plurality of electrode arrangements 7 are accommodated in the region of the channel widening 5, wherein the electrode arrangements 7 may also comprise measurement stations.

Furthermore, instead of the funnel-like electrode arrangement 3 (“funnel”), an electrode arrangement 3 is used as a distributor element which allows the loading of the electrode arrangements 7.

Moreover, in addition to the carrier flow channel 1, an additional loading channel 1′ opens into the region of the channel widening 5. In this example of embodiment, cells can firstly be trapped in the cage-like electrode arrangements 7 (“cages”) and then exposed to a spatial or temporal chemical concentration profile by suitably adjusting the flow conditions in the carrier flow channel 1 or loading channel 1′. This may involve for example supplying synthetic particles, pharmacological substances, antibodies, viruses, etc. via the loading channel 1′. Sorting can then take place in a manner depending on the detection. This example of embodiment advantageously combines low substance consumption with parallel evaluation in the microsystem.

The invention is not restricted to the preferred examples of embodiments described above. Rather, a large number of variants and modifications are possible which likewise make use of the inventive concept and therefore fall within the scope of protection.

### LIST OF REFERENCE NUMERALS

1 carrier flow channel
1′ loading channel
2 particle
3 electrode arrangement
4 electrode arrangement
5 channel widening
6 measurement station
7 electrode arrangement
8 output channel
9 output channel
10 electrode arrangement
11 electrode arrangement
12 sheath flow channel
13 sheath flow channel

1. A microfluidic system comprising at least one carrier flow channel for accommodating a carrier flow containing particles suspended therein, wherein the carrier flow channel has over part of its length at least one channel widening with a widened channel cross-section.

2. The microfluidic system according to claim 1, further comprising at least one measurement station for analyzing the particles suspended in the carrier flow, wherein the measurement station is arranged at least partially in a region of the channel widening.

3. The microfluidic system according to claim 2, further comprising a plurality of measurement stations for analyzing the particles suspended in the carrier flow, wherein at least one of the measurement stations is arranged in the region of the channel widening while at least one of the measurement stations is arranged outside the region of the channel widening.

4. The microfluidic system according to claim 1, further comprising at least one manipulation device for manipulating the suspended particles, wherein the manipulation device is arranged at least partially in a region of the channel widening.

5. The microfluidic system according to claim 4, wherein the channel widening is limited in a flow direction to a region of the manipulation device.

6. The microfluidic system according to claim 2, wherein the measurement station has a predefined maximum detection speed, up to which the measurement station can analyze the particles suspended in the carrier flow, wherein the carrier flow containing the particles suspended therein has a flow speed which is below the maximum detection speed in the region of the channel widening and above the maximum detection speed outside the channel widening.

7. The microfluidic system according to claim 6, wherein no field cage and no manipulation device is arranged in the region of the channel widening.

8. The microfluidic system according to claim 1, further comprising a field cage for fixing the suspended particles in the carrier flow channel, wherein the field cage is arranged in the region of the channel widening.

9. The microfluidic system according to claim 1, wherein the channel cross section of the carrier flow channel is widened by 10% to 500% in the region of the channel widening compared to the region outside the channel widening.

10. The microfluidic system according to claim 1, wherein the carrier flow channel branches into a plurality of output channels in a branching region located downstream behind the channel widening.

11. The microfluidic system according to claim 10, wherein a sorting device is arranged in the branching region, which sorting device sorts the suspended particles into one of the output channels depending on an actuation of the sorting device.

12. The microfluidic system according to claim 10, wherein a centering device is arranged in at least one of the output channels, which centering device centers the suspended particles in the output channel.

13. The microfluidic system according to claim 10, wherein at least one sheath flow channel opens into at least one of the output channels.

14. The microfluidic system according to claim 2, wherein a retaining device is arranged in the carrier flow channel upstream before the measurement station, which retaining device stops the suspended particles or allows them to pass, depending on its actuation.

15. The microfluidic system according to claim 14, wherein the sorting device, the centering device, the field cage and the retaining device comprise a dielectrophoretically acting electrode arrangement.

16. The microfluidic system according to claim 11, wherein the sorting device is arranged eccentrically in the carrier flow channel.

17. The microfluidic system according to claim 1, wherein the carrier flow channel is widened in just one spatial dimension in a region of the channel widening.

18. The microfluidic system according to claim 1, wherein the carrier flow channel is widened in two spatial dimensions in the region of the channel widening.

19. The microfluidic system according to claim 1, said microfluidic system being integrated on a chip.

20. The microfluidic system according to claim 1, wherein the channel widening consists of a splitting of the carrier flow channel into a plurality of parallel subchannels.

21. A cell sorter comprising a microfluidic system according to claim 1.

22-25. (canceled)

26. The microfluidic system according to claim 2, wherein the channel widening is limited in a flow direction to the region of the measurement station.

27. The microfluidic system according to claim 2, further comprising a field cage for fixing the suspended particles in the carrier flow channel, wherein the field cage is arranged at the measurement station.

28. The microfluidic system according to claim 2, wherein the measurement station is arranged eccentrically in the carrier flow channel.

29. A method for performing medical research comprising a use of the microfluidic system according to claim 1.

30. A method for performing pharmaceutical research comprising a use of the microfluidic system according to claim 1.

31. A method for performing diagnostics comprising a use of the microfluidic system according to claim 1.

32. A method for performing a forensic analysis comprising a use of the microfluidic system according to claim 1.

33. A method for separating different cell types from one another, said method comprising a use of the microfluidic system according to claim 1.

34. A method for forming pairs of cells comprising a use of the microfluidic system according to claim 1.

35. A method for performing cell fusion comprising a use of the microfluidic system according to claim 1.

36. A method for stretching cells comprising a use of the microfluidic system according to claim 1.

37. A method for collecting cells comprising using the microfluidic system according to claim 1 as a collection chamber.

38. A method for incubating cells comprising a use of the microfluidic system according to claim 1.
